(19) United States
(12) Patent Application Publication
(10) Pub. No.: US 2022/0288202 A1
(43) Pub. Date: Sep. 15, 2022

(54) Use of Calcineurin Inhibitor Free CTLA4-IG + Anti-IL6/IL6R For Long Term Immunosuppression in Solid Organ Transplant Recipients

(71) Applicant: Cedars-Sinai Medical Center, Los Angeles, CA (US)

(72) Inventors: Stanley C. Jordan, Manhattan Beach, CA (US); Ashley Vo, Northridge, CA (US); Noriko Ammerman, Los Angeles, CA (US); Edmund Huang, Redondo Beach, CA (US); Irene Kim, Los Angeles, CA (US)

(73) Assignee: Cedars-Sinai Medical Center, Los Angeles, CA (US)

(21) Appl. No.: 17/639,654

(22) PCT Filed: Sep. 4, 2020
(86) PCT No.: PCT/US2020/049422

§ 371 Date: Mar. 2, 2022

Related U.S. Application Data
(60) Provisional application No. 62/895,836, filed on Sep. 4, 2019.

(51) Int. Cl. A61K 39/395; A61P 37/06; A61K 38/17; A61K 38/13; A61K 31/436

(52) U.S. Cl. CPC A61K 39/3955; A61P 37/06; A61K 38/1774; A61K 38/13; A61K 31/436; A61K 2039/545

(57) ABSTRACT

The present invention provides for methods and uses of (i) an IL-6 inhibitor or IL-6R inhibitor, or both; and (ii) CT-LA-4 or CTLA-4 fusion proteins for immunosuppression and/or immunomodulation in a solid organ transplant recipient. Various embodiments of the method comprise administering an IL-6 inhibitor or IL-6R inhibitor, or both to the recipient; and administering a or CTLA-4 fusion protein such as CTLA4-Ig to the recipient and may further include administration or use of a calcineurin inhibitor.

### RELATED APPLICATIONS

This application claims priority to U.S. Provisional Application No. 62/895,836 filed on Sep. 4, 2019, the contents of which are incorporated by reference in their entirety.

### FIELD OF INVENTION

This invention relates to immunosuppression and immunomodulation in solid organ transplant recipients.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Development of alloantibodies post-transplant with subsequent antibody mediated rejection (ABMR) represents the major cause for allograft failure in the United States. An unmet medical need exists to improve our ability to develop novel, effective immunosuppressive protocols that would allow patients to maximally sustain life-saving organ transplants.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts an approach post-transplant immunosuppression using anti-IL-6 and CTLA4-Ig. After HLAi transplantation, patients will receive Campath-1H (anti-CD52) per standard protocol. Patients will also be started on tacrolimus with levels maintained in the 7-8 ng/ml range per center protocol. However, patients will not receive MMF but started on anti-IL-6 on day #7 post-transplant. This will be maintained until day #90 post-transplant when, if stable, patients will be slowly converted to CTLA4-Ig and tapered off tacrolimus as shown above. At day 180, patients will be off tacrolimus and maintained on anti-IL-6 25 mg administered subcutaneously (sq) monthly+CTLA4-Ig 5 mg/kg intravenously (iv) administered monthly. Patients will be monitored per center protocol for CMV, BK, EBV viral infections using PCR. We will also measure donor specific antibodies (DSAs), C-Reactive Protein (CRP), Treg cells, and dd-cfDNA as indicated. A protocol biopsy will be done at 12 M. Assessments will include standard pathology and molecular microscope analysis.

FIG. 2 shows the time course of DSAs, their impact on allograft histology and the eventual progression to allograft failure, leaving the recipient highly HLA sensitized.

FIG. 3 shows the course of treatment of a highly-HLA sensitized female patient who received an ABOi+HLAi kidney transplant. The treatment course was complicated by antibody rejection episodes and the patient was placed on chronic anti-IL-6R to control DSAs. As shown the patient's creatinine continued to rise with biopsy showing CNI toxicity three years after transplant. The patient was then treated with CTLA4-Ig+anti-IL-6R alone. As further shown after 2 years on this CTLA4-Ig+anti-IL-6R treatment regimen the DSAs in this patient were negative and the serum creatine (SCr) levels were found to have declined.

FIG. 4A shows the course of treatment of a 57 year old (y.o.) male 14 years and 3 months after receiving a transplant from a live donor. In 2016, the patient had an increase in SCr with biopsy showing evidence of ABMR and CNI toxicity. He was then treated with IVIg+rituximab followed by conversion to CTLA4-Ig. His course was stable until December 2017 when he developed a sharp rise in SCr. A biopsy showed evidence of chronic ABMR. He was then converted from CELLCEPT to tocilizumab monthly. As can be seen, he has been stable with declining SCr for over 1.5 years and continues on this regimen.

FIG. 4B shows the course of a patient transplanted with HLAi and DSA donor who had slow graft function with unacceptable nadir creatinine. Biopsy showed evidence of CNI toxicity. When patient was converted to CTLA4-Ig, SCr steadily improved and a biopsy obtained nearly 9M later showed no signs of rejection after the patient was maintained on Clazakizumab+CTLA4-Ig alone.

FIG. 5A-5B show the proportion of kidney transplant recipients who developed infections (all infections FIG. 5A, or serious infections FIG. 5B) post-transplant. Patients were in the low-risk group (non-Ritux #191) did not receive IVIG/rituximab or alemtuzumab, and (Ritux #171) received desensitization with rituximab, intravenous immunoglobulin (IVIG) followed by transplantation. Follow up for up to 45 months of these same patients shows similar infection rates. The most common infection was urinary tract infection in both groups. The incidence of CMV was greater in the non-Ritux group while there were more BK infections in the Rituximab group. No patients in the Rituximab group developed Post-Transplant Lymphoproliferative Disorders (PTLDs) or Progressive Multifocal Leukoencephalopathies (PMLs) while 2 patients in the non-Rituximab group developed PTLD42.

FIG. 6 shows the impact of clazakizumab on HLA antibodies (class I and class II). Significant reductions were seen that permitted all 10 claza-treated patients in this first cohort to be transplanted.

FIG. 7 shows that donor-specific antibodies present prior to transplant were significantly reduced at transplant and disappeared post-transplant in patients treated with clazakizumab.

FIG. 8 shows the significant reduction in pathogenic antibodies in patients treated with clazakizumab up to 3M post-transplant. Two of the ten transplanted patients had rejection episodes but no grafts were lost due to rejection.

FIG. 9 shows that the use of clazakizumab resulted in a significant increase in Treg cells which are associated with tolerance and graft acceptance in patients treated with clazakizumab. This dramatic increase may explain the absence of any evidence of rejection in all but 2 patients who were treated with clazakizumab post-transplant and no graft loss in any of the patients treated with clazakizumab.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, NY 2006); March, Advanced Organic Chemistry Reactions, Mechanisms and Structure 7t^{h} ed., J. Wiley & Sons (New York, NY 2013); and Sambrook and Russel, Molecular Cloning: A Laboratory Manual 4t^{h} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“ABO incompatible kidney” as used herein refers to a kidney where the donor blood type and the recipient blood type are different.

“Human leukocyte antigen incompatible kidney” (HLAi kidney) refers to a kidney where the transplant recipient has antibodies against the donor kidney.

“Highly-HLA sensitized” patient as used herein refers to a patient whose calculated panel reactive antigen (cPRA) is ≥50%.

It is important to note the multi-faceted ability of donor-specific antibodies (DSAs) to mediate allograft injury. Many of the pathologic features were once thought to be consequences of CNI toxicity leading to reduced dosing of these critical medications which further accelerated allograft loss. Emerging knowledge in this area is critical for development of newer techniques for suppression of DSA responses. What is important here is the persistence of the immune attack on the allograft. This eventually results in interstitial fibrosis and tubular atrophy (IF/TA), TG and allograft loss. Patients returning to dialysis have little hope of receiving a subsequent transplant and often face a higher risk of death on dialysis. DSAs are also known to accelerate atherosclerosis in the allograft thus hastening the vascular demise of the kidney. Thus strategies to eliminate or reduce DSAs after transplant are beneficial in extending the longevity of allografts.

We believe that the use of clazakizumab+CTLA4-Ig in highly-HLA sensitized patients presenting for incompatible kidney transplantation will provide a more robust and complete method for preventing dnDSA development and sustained allograft function without ABMR beyond that achieved with standard immunosuppression.

Embodiments herein include proposed initiation of anti-IL6+ tacrolinnus based immunosuppression at HLAi transplant. Patients will not receive mycophenolate mofetil (MMF) and will be monitored for 3 months. At 3 months, if stable, the patients will be transitioned from tacrolimus to CTLA4-Ig over a 3-month period. At 6 months post-transplant, all patients will be on CTLA4-Ig+anti-IL6, each given monthly. The primary end point of this open label study will be to assess donor specific antibody (dnDSA) development in this high-risk population. Protocol biopsies will be performed at 1 year. Patients will also be monitored using Donor-derived cell-free DNA (dd-cf-DNA) serially post-transplant.

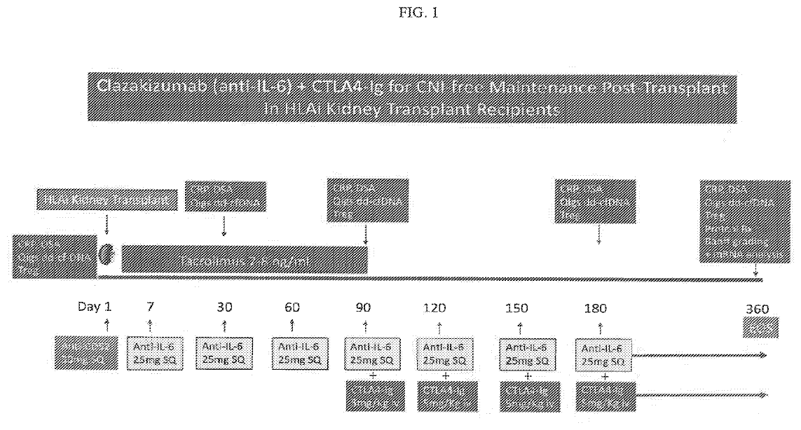

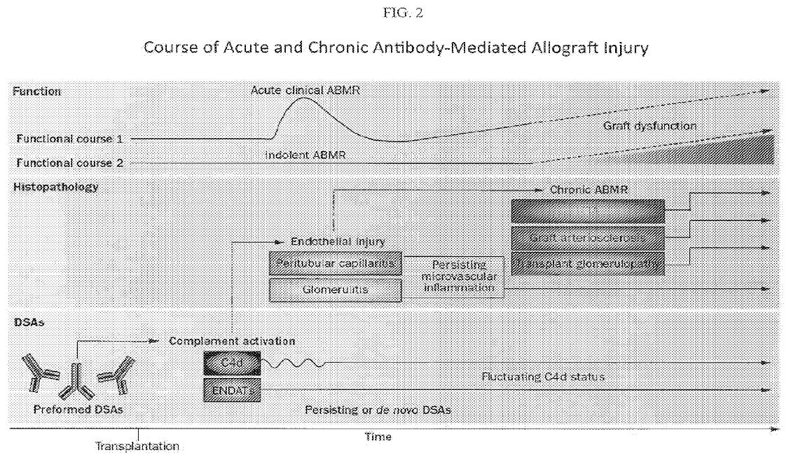

An exemplary protocol for post-transplant Clazakizumab+CTLA4-Ig maintenance immunosuppression is shown in FIG. 1. The planned end of study visit will be day #365. In the event significant benefits are seen with this protocol for the patients, consideration for continuation beyond the study end date will be given.

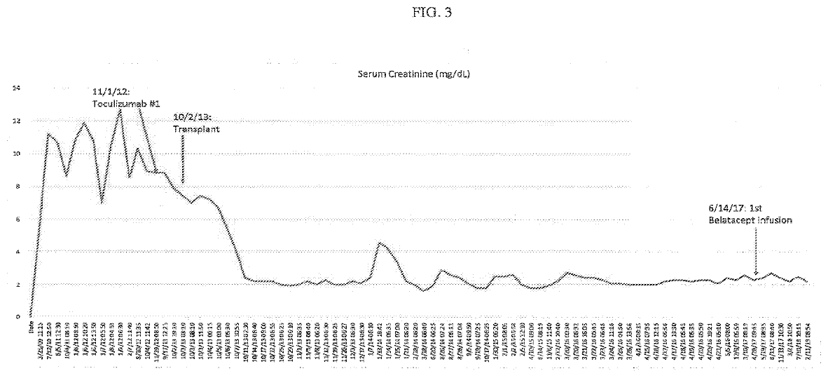

Antibodies to HLA antigens have a strong impact on mediation of allograft injury and loss and remain a persistent and often impenetrable barrier to successful transplantation for thousands of patients on renal transplant lists world wide. As depicted in FIG. 3, pre-formed or de novo DSAs activate complement, induce endothelial cell proliferation and mediate ADCC resulting in a progression of allograft dysfunction and loss. More than 5,000 renal allografts are lost each year in the U.S., approximately >50% to antibody mediated injury. Thus, understanding the pathophysiology of ABMR and B-cell activation are critical to improving the longevity of existing allografts and development of successful strategies to prevent ABM R. It is also imperative that we develop novel, effective therapies for prevention of allosensitizaton and ABMR since there are currently no FDA approved drugs for this condition or for treatment of ABM R.

A combination of CTLA4-Ig and anti-IL-6 or anti-IL-6R according to various embodiments (for example, combining the FDA approved drug, belatacept (CTLA4-Ig) with clazakizumab (anti-IL-6), as described in various embodiments herein), offers a powerful and synergistic approach to drastically improve standard immunosuppression and eliminating the need for the use of potentially toxic drugs such as tacrolimus and MMF. Current toxicities of standard of care immunosuppression have several drawbacks. These include a considerable pill burden, persistent toxicities to the kidney, nervous system and gastrointestinal tract and a high risk for medication non-adherence which puts patients at substantial risk for dnDSA development and ABMR.

Data from animal models and our own patient population show that combining anti-IL-6 with CTLA4-Ig should provide a less toxic and more effective post-transplant immunosuppressive and/or immunomodulatory protocol and moreover should result in much higher patient compliance. In addition, limiting allosensitization will improve long-term outcomes for these patients.

In summary, the combination of CTLA4-Ig and anti-IL-6 or anti-IL-6R according to various embodiments of the present invention will replace the need for standard immunosuppression resulting in better adherence and long term graft and patient survival. This combination will result in a more robust and complete protocol for preventing dnDSA development in this high-risk population and sustained allograft function without ABMR much beyond what is currecntly achievable with standard immunosuppression.

As such, various embodiments of the present invention provide for a method of immunosuppression or immunomodulation in a solid organ transplant recipient, comprising: administering an IL-6 inhibitor or IL-6R inhibitor, or both to the recipient; and further administering CTLA4-Ig to the recipient, wherein these moieties may be administered separately or in combination.

Also, various further embodiments of the present invention provide IL-6 inhibitor or IL-6R inhibitor for use in immunosuppression or immunomodulation in a solid organ transplant recipient, comprising: administering an IL-6 inhibitor or IL-6R inhibitor, or both to the recipient; and administering a CTLA4-Ig to the recipient.

In various embodiments, a first dose of the IL-6 inhibitor or the IL-6R inhibitor is administered about 5-10 days after transplantation; for example, 5, 6, 7, 8, 9 or 10 days after transplantation. In a particular embodiment, the first dose of the IL-6 inhibitor or the IL-6R inhibitor is administered about 7 days after transplantation. Thereafter, the IL-6 inhibitor or the IL-6R inhibitor is administered every 20-40, 20-30, 20-25, 25-30, 30-35, 35-40 days or every 3-5 weeks after transplantation; for example, every 20, 25, 30, 35, or 40 days, or every 3, 4, or 5 weeks. In a particular embodiment, the IL-6 inhibitor or the IL-6R inhibitor is administered about every 30 days.

In various embodiments, the IL-6 inhibitor or the IL-6R inhibitor is administered for at least one year. In various embodiments, the IL-6 inhibitor or the IL-6R inhibitor is administered for at least 2, 3, 4, 5, 6, 7, 8, 9, or 10 years. In various embodiments, the IL-6 inhibitor or the IL-6R inhibitor is administered indefinitely.

In various embodiments, a first dose of the CTLA4-Ig is administered about 75-105, 75-85, 75-80, 80-85, 85-95, 85-90, 90-100, 90-95, 95-100, or 100-105 days after transplantation; for example, 75, 80, 85, 90, 95, 100 or 105 days after transplantation. In particular embodiments, the first dose of the CTLA4-Ig is administered about 90 days after transplantation.

In various embodiments, where the transplant recipient is administered a calcineurin inhibitor, the first dose of the CTLA4-Ig is administered after ending the administration of the calcineurin inhibitor. In various embodiments, where the transplant recipient is administered a calcineurin inhibitor, the first dose of the CTLA4-Ig is administered after lowering the dosage of the calcineurin inhibitor. After the first dose of the CTLA4-Ig, the subsequent doses of CTLA4-Ig are administered about every 20-40, 20-30, 30-40, 20-25, 25-30, 30-35, 35-40 days or every 3-5 weeks; for example, every 20, 25, 30, 35, or 40 days, or every 3, 4, or 5 weeks. In a particular embodiment, the CTLA4-Ig is administered about every 30 days.

In various embodiments, the CTLA4-Ig is administered for at least one year. In various embodiments, the CTLA4-Ig is administered for at least 2, 3, 4, 5, 6, 7, 8, 9, or 10 years. In various embodiments, the CTLA4-Ig is administered indefinitely.

In various embodiments, the method further comprises administering a calcineurin inhibitor 0-3 days after transplantation (e.g., day 0, or 1, 2 or 3 days after transplantation) and continuing to administer the calcineurin inhibitor about 75-105, 75-85, 75-80, 80-85, 80-90, 85-90, 90-100, 90-95, 95-100, or 100-105 days (e.g., 75, 80, 85, 90, 95, 100, or 105 days) after transplantation.

In various embodiments, the method comprises administering a calcineurin inhibitor 0-3 days after transplantation (e.g., day 0, or 1, 2 or 3 days after transplantation) and stopping the administration of the calcineurin inhibitor after about 75-105, 75-85, 75-80, 80-85, 80-90, 85-90, 90-100, 90-95, 95-100, or 100-105 days (e.g., 75, 80, 85, 90, 95, 100, or 105 days) after transplantation.

In various embodiments, the method does not comprise administering a calcineurin inhibitor; that is, the recipient does not receive a calcineurin.

In various embodiments, the method prevents or reduces the likelihood of ABMR.

In various embodiments, the method prevents or reduces the likelihood of dnDSA development.

In various embodiments, the method prevents or reduces the likelihood of allosensitization.

In various embodiments, the IL-6 inhibitor is clazakizumab, and the CTLA4-Ig is belatacept. In these embodiments methods of immunosuppression or immunomodulation are provided in a solid organ transplant recipient comprising: administering a first dose of clazakizumab about 5-10 (5, 6, 7, 8, 9 or 10) days after transplantation; administering a subsequent dose of clazakizumab every 20-40 days for at least one year; administering a first dose of belatacept about 75-105, 85-95, 75-85, 75-80, 80-85, 80-90, 85-90, 90-100, 90-95, 95-105, 95-100, or 100-105 days after transplantation; and administering a subsequent dose of belatacept every 20-40, 20-30, 20-25, 20-30, 25-30, 30-35 or 35-40 days.

In particular embodiments, the method of immunosuppression or immunomodulation in a solid organ transplant recipient comprises: administering a first dose of clazakizumab about 7 days after transplantation; administering a subsequent dose of clazakizumab about every 30 days; administering a first dose of belatacept about 90 days after transplantation; and administering a subsequent dose of belatacept about every 30 days. In various embodiments, the method does not comprise administering a calcineurin inhibitor after 75-105, 75-85, 75-80, 80-85, 80-90, 85-90, 90-100, 90-95, 95-100, or 100-105 days after transplantation.

### Kits

The present invention is also directed to kits for immunosuppression or immunomodulation. The kits are useful for practicing the inventive method of immunosuppression or immunomodulation. The kits comprise an assemblage of materials or components, including at least one of the inventive compositions. Thus, in some embodiments the kit will contain a composition including an IL-6 inhibitor or IL-6R inhibitor, or both; and a composition comprising CTLA4-Ig; as described herein.

The exact nature of the components configured in the inventive kit depends on its intended purpose. For example, some embodiments are configured for the purpose of immunosuppression or immunomodulation in a solid organ transplant recipient; for example, a kidney transplant recipient. In one embodiment, the kit is configured particularly for the purpose of immunosuppression or immunomodulation in non-human mammalian subjects. In another embodiment, the kit is configured particularly for the purpose of immunosuppression or immunomodulation in human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such for immunosuppression or immunomodulation of a transplant recipient. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in immunosuppression or immunomodulation. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing an IL-6 inhibitor or IL-6R inhibitor, or both; and a CTLA4-Ig. The packaging material generally has an external label which indicates the contents and/or purpose of the kit and/or its components.

Various embodiments of the invention provide for a kit for immunosuppression or immunomodulation, comprising: an IL-6 inhibitor or IL-6R inhibitor, or both; and a CTLA4-Ig; and instructions for using the IL-6 inhibitor or IL-6R inhibitor, or both, and the CTLA4-Ig for immunosuppression or immunomodulation in a solid organ transplant recipient.

In various embodiments, the IL-6 inhibitor or IL-6R inhibitor is provided in specific measured quantities or doses. Examples include, but are not limited to 5-50, 10-50, 10-45, 10-40, 10-35, 10-30, 10-25, 5-10, 10-20, 5, 10, 15, 20, 25, 30, 35, 40, 45, 50 mg dosages. In particular embodiments, the IL-6 inhibitor or IL-6R inhibitor is provided in 25 mg or 15 mg dosages.

In various embodiments, the CTLA4-Ig is provided in measured quantities. Examples include but are not limited to 50-600, 100-500, 100-400, 150-200, 150-250, 150-300, 150-400, 100, 150, 200, 250, 300, 350, 400, 450, or 500 mg for dosing according to the weight of the recipient, wherein these dosage amounts may be administered in one or multiple dosage forms.

### CTLA-4 or CTLA-4 Fusion Proteins, Preferably CTLA4-Ig

The invention includes methods or uses including the administration of CTLA-4 or a CTLA-4 fusion protein. CTLA-4 also known as CD152 (cluster of differentiation 152), is a protein receptor polypeptide that functions as an immune checkpoint and downregulates immune responses. CTLA-4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells.

CTLA-4 fusion proteins herein include any CTLA-4 polypeptide fused to another polypeptide or in vivo half-life extender, e.g., an Ig, annexin, XTEN, PAS, or serum albumin, natural and semi-synthetic polysaccharides, including O- and N-linked oligosaccharides, dextran, hydroxyethyl starch (HES), polysialic acid and hyaluronic acid, as well as unstructured protein polymers such as homo-amino acid polymers, and elastin-like polypeptides, Most typically the CTLA-4 fusion will comprise a CTLA4-Ig polypeptide, which in general comprises a fusion protein having an extracellular domain (or a modified extracellular domain) of CTLA-4 fused to a portion of the Fc domain of an immunoglobulin (e.g., IgG, typically a human IgG); examples of CTLA4-Ig fusions include but are not limited to, belatacept, belatacept biosimilar and abatacept.

CTLA4-Ig used in the various embodiments of the present invention can be selected from the group consisting of abatacept, belatacept, belatacept biosimilar (KN-019), salts thereof and combinations thereof.

### IL-6 Inhibitor

An IL-6 inhibitor includes any compound, generally an antibody, which binds to IL-6 and antagonizes (blocks or inhibits) its in vivo effects. Exemplary IL-6 inhibitors used in the various embodiments of the present invention can be an IL-6 inhibitor antibody or compound selected from the group consisting of siltuximab, clazakizumab, olokizumab, sirukumab, FB-704A, ARGX-109, EBI-031, AH-65, SL-1026, ES-306, AM-201, Isilimomab (also known as “B-E8”), MAb 1339 (a high affinity variant of Elsilimomab), salts thereof and combinations thereof. The amino acid sequence of clazakizumab is well known in the art and is disclosed e.g., in W02019136266 which PCT is incorporated by reference herein. In this PCT clazakizumab comprises the heavy chain polypeptide of SEQ ID NO: 704 and the light chain polypeptide of SEQ ID NO: 702.

### IL-6R Inhibitor

An IL-6R inhibitor includes any compound, generally an antibody, which binds to IL-6R and antagonizes (blocks or inhibits) its in vivo effects. Exemplary IL-6R inhibitors used in the various embodiments of the present invention can be an IL-6R inhibitor antibody or compound selected from the group consisting of tocilizumab, sarilumab, tocilizumab biosimilar (BAT-1806), Satralizumab, vobarilizumab, olamkicept, BCD-089, CMAB-806, tocilizumab biosimilar (QX-0035), HS-628, tocilizumab biosimilar (LusiNEX), MT-6194, TZLS-501, salts thereof and combinations thereof.

### Calcineurin Inhibitor

Calcineurin inhibitors comprise a class of drugs which target and block or inhibit the effects of calcineurin. Calcineurin (CaN) is a calcium and calmodulin dependent serine/threonine protein phosphatase (also known as protein phosphatase 3, and calcium-dependent serine-threonine phosphatase) which activates T cells. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), which, in turn, stimulates the growth and differentiation of the T cell response. Exemplary calcineurin inhibitors which may be used in the various embodiments of the present invention can be selected from the group consisting of cyclosporine, cyclosporine (modified), voclosporin, pimecrolimus, tacrolimus, salts thereof and combinations thereof.

### Transplant Recipient

In various embodiments of the present invention, the recipient is a highly-HLA sensitized patient.

In various embodiments, the recipient is a solid organ transplant recipient, e.g., a heart, lung, liver, pancreas, small intestine, thymus, or a combination of any of the foregoing solid organs transplant recipient. In various embodiments, the recipient is a solid organ transplant recipient of an incompatible solid organ. In a particular embodiment, the recipient is solid organ transplant recipient of an HLA incompatible solid organ. In some instances the organ may be from a living donor. In other instances the organ may be obtained from a deceased donor or non-human donor.

In various embodiments, the recipient is a kidney transplant recipient. In various embodiments, the recipient is a kidney transplant recipient of an incompatible kidney. In a particular embodiment, the recipient is kidney transplant recipient of an HLA incompatible kidney.

### Solid Organ

In various embodiments, the solid organ is a kidney. In various embodiments, the kidney is an incompatible kidney (e.g., ABOi or HLAi).

In various embodiments, the solid organ is heart, lung, liver, pancreas, small intestine, or thymus. In various embodiments, the solid organ is an incompatible heart, lung, liver, pancreas, small intestine, or thymus (e.g., ABOi or HLAi).

In various embodiments, the present invention provides pharmaceutical compositions including a pharmaceutically acceptable excipient along with a therapeutically effective amount of IL-6 inhibitor or IL-6R inhibitor, or both, and a CTLA-4 or CTLA-4 fusion, e.g., CTLA4-Ig. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or lyophilized form for reconstitution prior to injection, and particularly for subcutaneous injection.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

### Exemplary Embodiments

The following exemplary embodiments are provided to better illustrate the claimed invention and its applications.

### Embodiments

1. A method of immunosuppression or immunomodulation in a solid organ transplant recipient, comprising:

administering an IL-6 inhibitor or IL-6R inhibitor, or both to the recipient; and

administering CTLA-4 or a CTLA4 fusion protein, preferably CTLA4-Ig, to the recipient.
2. An IL-6 inhibitor or IL-6R inhibitor and a CTLA-4 or a CTLA-4 fusion protein, preferably

CTLA-4-Ig, for use in immunosuppression or immunomodulation in a solid organ transplant recipient, wherein the use comprises administering an IL-6 inhibitor or IL-6R inhibitor, or both to the recipient and administering a CTLA4-Ig to the recipient.
3. The method or use of Embodiment 1 or 2, wherein the CTLA-4 or a CTLA4 fusion protein comprises

(i) belatacept;

(ii) belatacept biosimilar;

(iii) abatacept; or

(iv) any combination of the foregoing.
4. The method or use of Embodiment 1, 2 or 3, wherein the IL-6 inhibitor comprises:

(i) Siltuximab;

(ii) Clazakizumab;

(iii) Olokizumab;

(iv) sirukumab;

(v) FB-704A;

(vi) ARGX-109;

(vii) EBI-031;

(viii) AH-65;

(ix) SL-1026;

(x) ES-306;

(xi) AM-201;

(xii) Isilimomab (also known as “B-E8”);

(xiii) MAb 1339 (a high affinity variant of Elsilimomab);

(xiv) a salt of any of the foregoing; or

(xv) any combination of the foregoing.
5. The method or use of Embodiment 1, 2, 3 or 4, wherein the IL-6-R inhibitor comprises:

(i) Tocilizumab;

(ii) Sarilumab;

(iii) tocilizumab biosimilar (BAT-1806);

(iv) Satralizumab;

(v) Vobarilizumab;

(vi) Olamkicept;

(vii) BCD-089;

(viii) CMAB-806;

(ix) tocilizumab biosimilar (QX-003S);

(x) HS-628;

(xi) tocilizumab biosimilar (LusiNEX);

(xii) MT-6194;

(xiii) TZLS-501;

(xiv) a salt of any of the foregoing; or

(xv) any combination of the foregoing.
6. The method or use of any one of Embodiments 1-5, wherein a first dose of the IL-6 inhibitor and/or the IL-6R inhibitor is administered about 5-10 days after transplantation.
7. The method of any one of Embodiments 1-5, wherein IL-6 inhibitor and/or the IL-6R inhibitor is administered every 20-40 days after transplantation.
8. The method or use of any of the above Embodiments, wherein the IL-6 inhibitor and/or the IL-6R inhibitor is administered for at least one year.
9. The method or use of any of Embodiments 1-8, wherein a first dose of the CTLA-4 or CTLA-4 fusion protein, preferably CTLA4-Ig is administered about 75-105 days after transplantation.
10. The method or use of any of Embodiments 1-8, wherein the CTLA-4 or CTLA-4 fusion protein, preferably CTLA4-Ig is administered about every 20-40 days after the first dose of the CTLA4-Ig.
11. The method or use of any of the above Embodiments, wherein the CTLA-4 or CTLA-4 fusion protein, preferably CTLA4-Ig is administered for at least one year.
12. The method or use of any of the above Embodiments, further comprising administering a calcineurin inhibitor.
13. The method of any of the above Embodiments, further comprising administering a calcineurin inhibitor selected from the group consisting of

(i) cyclosporine;

(ii) cyclosporine (modified);

(iii) Voclosporin;

(iv) Pimecrolimus;

(v) tacrolimus;

(vi) a salt of any of the foregoing;

(vii) any combination of the foregoing.
14. The method or use of Embodiment 12 or 13, wherein the calcineurin inhibitor is administered 0-3 days after transplantation and continuing to administer the calcineurin inhibitor for about 75-105 days after transplantation.
15. The method or use of any of the above Embodiments, further comprising administering a calcineurin inhibitor 0-3 days after transplantation and stopping the administration of the calcineurin inhibitor after about 75-105 days after transplantation.
16. The method or use of any of Embodiments 1-11, wherein the method or use does not comprise administering a calcineurin inhibitor.
17. The method or use of any of the above Embodiments, wherein:

(i) the IL-6 inhibitor is clazakizumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(ii) the IL-6 inhibitor is Siltuximab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(iii) the IL-6 inhibitor is olokizumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(iv) the IL-6 inhibitor is sirukumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(v) the IL-6 inhibitor is FB-704A; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(vi) the IL-6 inhibitor is ARGX-109; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(vii) the IL-6 inhibitor is EBI-031; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(viii) the IL-6 inhibitor is AH-65; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(ix) the IL-6 inhibitor is ES-306; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(x) the IL-6 inhibitor is AM-201; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(xi) the IL-6 inhibitor is Isilimomab (also known as “B-E8”); the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(xii) the IL-6 inhibitor is MAb 1339 (a high affinity variant of Elsilimomab); the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(xiii) the IL-6 inhibitor is a salt of any of the foregoing; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors; or

(xiv) the IL-6 inhibitor is any combination of the foregoing IL-6 inhibitors; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors.
18. The method or use of any of the above Embodiments, wherein:

(i) the IL-6-R inhibitor is Tocilizumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(ii) the IL-6-R inhibitor is Sarilumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(iii) the IL-6-R inhibitor is tocilizumab biosimilar (BAT-1806); the CTLA4-Ig is belatacept, belatacept biosimilar, or abatacept, preferably belatacept; and the calcineurin inhibitor, if administered, is selected from the group consisting of cyclosporine, cyclosporine, cyclosporine (modified), voclosporin, pimecrolimus, tacrolimus, a salt of any of the foregoing, or any combination of the foregoing calcineurin inhibitors;

(iv) the IL-6-R inhibitor is Satralizumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(v) the IL-6-R inhibitor is Vobarilizumab; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(vi) the IL-6-R inhibitor is Olamkicept; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(vii) the IL-6-R inhibitor is BCD-089; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(viii) the IL-6-R inhibitor is CMAB-806, the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(ix) the IL-6-R inhibitor is tocilizumab biosimilar (QX-003S); the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(x) the IL-6-R inhibitor is HS-628; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(xi) the IL-6-R inhibitor is tocilizumab biosimilar (LusiNEX); the CTLA4-Ig is belatacept, belatacept biosimilar, or abatacept, preferably belatacept; and the calcineurin inhibitor, if administered is selected from the group consisting of cyclosporine, cyclosporine (modified), voclosporin, pimecrolimus, tacrolimus, a salt of any of the foregoing, or any combination of the foregoing calcineurin inhibitors;

(xii) the IL-6-R inhibitor is MT-6194; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors;

(xiii) the IL-6-R inhibitor is TZLS-501; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors; or

(xiv) the IL-6-R inhibitor is a salt of any of the foregoing Il-6R inhibitors; the CTLA4-Ig is belatacept, belatacept biosimilar, or abatacept, preferably belatacept; and the calcineurin inhibitor, if administered, is selected from the group consisting of cyclosporine, cyclosporine (modified), voclosporin, pimecrolimus, tacrolimus, a salt of any of the foregoing, or any combination of the foregoing calcineurin inhibitors.
19. The method or use of any of the above Embodiments, wherein the recipient is a highly-HLA sensitized recipient.
20. The method or use of any of the above Embodiments, wherein the solid organ comprises a kidney, heart, lung, pancreas, liver, or a combination of any of the foregoing.
21. The method or use of any of the above Embodiments, wherein the solid organ comprises a kidney.
22. The method or use of Embodiment 19 or 20, wherein the solid organ, optionally a kidney is human leukocyte antigen (HLA) incompatible.
23. The method or use of any of the above Embodiments, comprising preventing or reducing the likelihood of ABMR.
24. The method or use of any of the above Embodiments, comprising preventing or reducing the likelihood of dnDSA development.
25. The method or use of any of the above Embodiments, comprising preventing or reducing the likelihood of allosensitization.
26. The method or use of any of the above Embodiments, wherein the recipient is a highly-HLA sensitized recipient.
27. A method of immunosuppression or immunomodulation in a solid organ transplant recipient, comprising:

administering a first dose of clazakizumab about 5-10 days after transplantation to the recipient;

administering a subsequent dose of clazakizumab every 20-40 days to the recipient;

administering a first dose of belatacept about 75-105 days after transplantation to the recipient; and

administering a subsequent dose of belatacept every 20-40 days to the recipient.
28. An IL-6 inhibitor and a CTLA-4 fusion protein, for use in immunosuppression or immunomodulation in a solid organ transplant recipient, wherein the IL-6 inhibitor comprises and the CTLA-4 fusion protein comprises belatacept comprising:

administering a first dose of clazakizumab about 5-10 days after transplantation to the recipient;

administering a subsequent dose of clazakizumab every 20-40 days to the recipient;

administering a first dose of belatacept about 75-105 days after transplantation to the recipient; and

administering a subsequent dose of belatacept every 20-40 days to the recipient.
29. The method or use of Embodiment 27 or 28, wherein the method or use does not comprise administering a calcineurin inhibitor after 75-105, 75-95, 75-85, or 95-105 days after transplantation.
30. The method or use of any of the foregoing Embodiments wherein ANC (absolute neutrophil count) (cells/mm³) is monitored and dosing of the IL-6 or IL-6R inhibitor, e.g., clazakizumab is maintained if ANC is >1000.
31. The method or use of any of the foregoing Embodiments wherein ANC (absolute neutrophil count) (cells/mm³) is monitored and dosing of the IL-6 or IL-6R inhibitor, e.g., clazakizumab is interrupted if ANC ranges from 500-1000.
32. The method or use of Embodiment 31, wherein ANC (absolute neutrophil count) (cells/mm³) is monitored and dosing of the IL-6 or IL-6R inhibitor, e.g., clazakizumab is resumed if ANC is 1000 or more.
33. The method or use of any of the foregoing Embodiments wherein ANC (absolute neutrophil count) (cells/mm³) is monitored and dosing of the IL-6 or IL-6R inhibitor, e.g., clazakizumab is discontinued if ANC is less than 500.
34. The method or use of any of the foregoing Embodiments wherein platelet count (cells/mm³) is monitored and dosing of the IL-6 or IL-6R inhibitor, e.g., clazakizumab is maintained if Platelet count (cells/mm³) is >100,000.
35. The method or use of any of the foregoing Embodiments wherein platelet count (cells/mm³) is monitored and dosing of the IL-6 or IL-6R inhibitor, e.g., clazakizumab is interrupted if platelet count is within the range of 50,000-100,000.
36. The method or use of Embodiment 34 wherein platelet count (cells/mm³) is monitored and dosing of the IL-6 or IL-6R inhibitor, e.g., clazakizumab, is resumed if platelet count increases to >100,000, e.g., at 25 mg SQ monthly.
37. The method or use of any of the foregoing Embodiments wherein platelet count (cells/mm³) is monitored and dosing of the IL-6 or IL-6R inhibitor, e.g., clazakizumab is discontinued if platelet count is <50,000.
38. The method or use of any of the foregoing Embodiments wherein liver enzyme levels (ALT and AST) are monitored and dosing of the IL-6 or IL-6R inhibitor, e.g., clazakizumab, is maintained if elevations in liver enzymes (ALT and AST) do not increase to >3 to 5× ULN or do not persist in the range (>1 to 3× ULN).
39. The method or use of any of the foregoing Embodiments wherein liver enzyme levels (ALT and AST) are monitored and if liver enzyme levels increase >1 to 3× ULN, the dosage of concomitant transplant immunosuppressive drugs the subject is receiving is modified (increased) and/or at least one other concomitant transplant immunosuppressive drug is administered.
40. The method or use of Embodiment 39, wherein if said elevations in liver enzymes (ALT and AST) persist in the range (>1 to 3× ULN) then dosing of the IL-6 or IL-6R inhibitor, e.g., clazakizumab is interrupted until ALT/AST levels have normalized.
41. The method or use of any of the foregoing Embodiments wherein if elevations in liver enzymes (ALT and AST) increase in the recipient to within the range of >3 to 5× ULN, optionally confirmed by at least 2 tests, dosing of the IL-6 or IL-6R inhibitor, e.g., clazakizumab in the recipient is interrupted until liver enzymes are <3× ULN.
42. The method or use of Embodiment 41, wherein when elevations in liver enzymes (ALT and AST) are reduced to within the range of >1 to 3× ULN the dosage of concomitant transplant immunosuppressive drugs the subject is receiving is modified (increased) and/or at least one other concomitant transplant immunosuppressive drug is administered.
43. The method or use of any of the foregoing Embodiments, wherein liver enzyme and total bilirubin in the recipient are monitored and if the recipient experiences (i) persistent increases in liver enzymes of >3× ULN (ii) liver enzyme increases of >5× ULN and/or (iii) if total bilirubin is >2 × ULN then administration of the IL-6 or IL-6R inhibitor, e.g., clazakizumab is discontinued in the recipient.
44. The method or use of any of the foregoing Embodiments wherein the anti-IL6 and/or anti-IL6R inhibitor/CTLA-4 dosage regimen comprises 2 or optionally 3 distinct phases (i) an initial phase where anti-IL6 or anti-IL6R inhibitor is administered, without any CTLA-4 or CTLA-4 fusion protein, e.g., CTLA4-Ig, (2) a second phase after the initial phase where both anti-IL6/anti-IL6R inhibitor and CTLA-4 or CTLA-4 fusion protein, e.g., CTLA4-Ig are administered, and optionally (3) a third phase after (2) where only anti-IL6/anti-IL6R inhibitor or CTLA-4 or CTLA-4 fusion protein, e.g., CTLA4-Ig is administered.
45. The method or use of Embodiment 44 wherein the anti-IL-6 and/or anti-IL-6R inhibitor/CTLA-4 dosage regimen does not include administration of a calcineurin inhibitor.
46. The method or use of Embodiment 44 or 45 wherein (i) Phase 1 comprises administering a first dose of anti-IL6 or anti-IL6R inhibitor, e.g., clazakizumab about 5-10 (5, 6, 7, 8, 9 or 10) days after transplantation and another dose about every 20-40 days; and (ii) Phase 2 comprises administering a first dose of belatacept about 75-105, 85-95, 75-85, 75-80, 80-85, 80-90, 85-90, 90-100, 90-95, 95-105, 95-100, or 100-105 days after transplantation and administering a subsequent dose of belatacept every 20-40, 20-30, 20-25, 20-30, 25-30, 30-35 or 35-40 days and further includes administration of the anti-IL6 or anti-IL6R inhibitor, e.g., clazakizumab about every 20-40 days.
47. The method or use of Embodiments 44-46 wherein (i) Phase 1 comprises administering a first dose of clazakizumab about 7 days after transplantation; administering a subsequent dose of clazakizumab about every 30 days; and (ii) Phase 2 comprises administering a first dose of belatacept about 90 days after transplantation; and administering a subsequent dose of belatacept about every 30 days.
48. The method or use of any of Embodiments 44-47 which does not include administering a calcineurin inhibitor after 75-105, 75-85, 75-80, 80-85, 80-90, 85-90, 90-100, 90-95, 95-100, or 100-105 days after transplantation.
49. The method or use of any of Embodiments 44-48 wherein the anti-IL-6 and/or anti-IL-6R inhibitor/CTLA-4 dosage regimen dosage regimen includes the administration of a calcineurin inhibitor, optionally in phase (2) and/or (3).
50. The method or use of any of the foregoing Embodiments wherein the anti-IL-6 and/or anti-IL-6R inhibitor comprises clazakizumab and the CTLA-4 comprises CTLA-4-Ig, optionally belatacept, belatacept biosimilar, or abatacept, and preferably belatacept.
51. The method or use of any of the foregoing Embodiments wherein if a calcineurin inhibitor is administered, it is optionally selected from the group consisting of cyclosporine, cyclosporine (modified), tacrolimus, a salt of any of the foregoing, or any combination of the foregoing calcineurin inhibitors.
52. A kit for immunosuppression or immunomodulation, comprising:

an IL-6 inhibitor or IL-6R inhibitor, or both; and

a CTLA4-Ig; and

instructions for using the IL-6 inhibitor or IL-6R inhibitor, or both, and the CTLA4-Ig for immunosuppression or immunomodulation in a solid organ transplant recipient.
53. The kit of Embodiment 52, wherein the CTLA4-Ig comprises:

(i) belatacept,

(ii) belatacept biosimilar,

(iii) abatacept, or

(iv) any combination of the foregoing.
54. The kit of Embodiment 52, wherein the CTLA4-Ig comprises belatacept.
55. The kit of any of Embodiments 52-54, wherein the IL-6 inhibitor comprises:

(i) Siltuximab;

(ii) Clazakizumab,

(iii) olokizumab,

(iv) sirukumab,

(v) FB-704A,

(vi) ARGX-109,

(vii) EBI-031,

(viii) AH-65,

(ix) SL-1026,

(x) ES-306,

(xi) AM-201,

(xii) Isilimomab (also known as “B-E8”),

(xiii) MAb 1339 (a high affinity variant of Elsilimomab);

(xiv) a salt of any of the foregoing; or

(xv) any combination of the foregoing,
56. The kit of any of Embodiments 52-55, wherein the IL-6-R inhibitor comprises:

(i) Siltuximab;

(ii) Clazakizumab,

(iii) olokizumab,

(iv) sirukumab,

(v) FB-704A,

(vi) ARGX-109,

(vii) EBI-031,

(viii) AH-65,

(ix) SL-1026,

(x) ES-306,

(xi) AM-201,

(xii) Isilimomab (also known as “B-E8”),

(xiii) MAb 1339 (a high affinity variant of Elsilimomab);

(xiv) a salt of any of the foregoing; or

(xv) any combination of the foregoing. 57. The kit of any of Embodiments 52-56, which also includes a calcineurin inhibitor, optionally selected from the group consisting of cyclosporine, cyclosporine (modified), voclosporin, pimecrolimus, tacrolimus, a salt of any of the foregoing, or any combination of the foregoing calcineurin inhibitors.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Experience with Anti-IL-6/IL-6R+CTLA4-Ig in Human Kidney Transplant Recipients

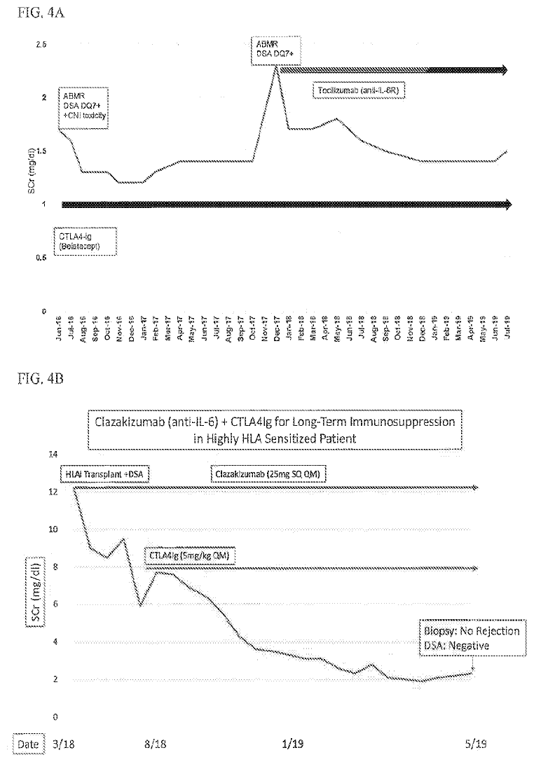

Four patients were maintained on CNI-free and MMF-free immunosuppression on anti-IL-6R or anti-IL-6+CTLA4-Ig for a prolonged time period (years) after transplant. These patients received two infusions per month as their standard immunosuppression. Data for three of these patients who had long-term follow up are shown in FIG. 3, FIG. 4A and FIG. 4B.

One of these 4 patients was transplanted in 2013 with an ABOi+HLAi kidney transplant and developed episodes of ABMR early post-transplant that were treated with PLEX+IVIg+Rituximab (FIG. 3). She subsequently was placed on anti-IL-6R with reduction in CELLCEPT. Three years later she was converted to CTLA4-Ig and tapered off tacrolimus due to toxicity seen on a biopsy. 6 years post-transplant she was found to be-negative DSA and to have serum Cr levels similar to that seen when she was initially transplanted. 7 years post-transplant she still is off CELLCEPT and tacrolimus.

FIG. 4A shows the course of treatment of a 57 year old (y.o.) male now 14 years 3M after receiving a transplant from a live donor. In 2016, the patient had an increase in SCr with biopsy showing evidence of ABMR and CNI toxicity. He was then treated with IVIg+rituximab followed by conversion to CTLA4-Ig. His course was stable until 12/2017 when he developed a sharp rise in SCr. A biopsy showed evidence of chronic ABMR. He was then converted from CELLCEPT to tocilizumab monthly. As can be seen, he has been stable with declining SCr for over 1.5 years and continues on this regimen.

FIG. 4B shows the course of a patient transplanted with HLAi and DSA+donor who had slow graft function with unacceptable nadir creatinine. Biopsy showed evidence of CNI toxicity. When this patient was converted to CTLA4-Ig, SCr levels steadily improved and a biopsy obtained nearly 9M later showed no signs of rejection after the patient was maintained on Clazakizumab+CTLA4-Ig alone.

The above 3 patients whose results post-treatment are discussed herein and further shown in in FIG. 3, FIG. 4A and FIG. 4B represent cases of very highly HLA sensitized patients who underwent HLAi transplant but showed unacceptable renal function with signs of CNI toxicity. All were converted to CTLA4-Ig, two in combination with tocilizumab (anti-IL-6R) and one with clazakizumab (anti-IL-6). Two of these three patients have exhibited excellent results with long term biopsies and showed no evidence of rejection years after transplant. The third patient was found to be stable years after transplant and has not required another biopsy.

### Example 2

### Assessing Safety and Efficacy of clazakizumab+CTLA4-Ig in preventing dnDSA development and ABMR post-HLAi transplant

This study is an open label design to further assess the safety and efficacy of clazakizumab+CTLA4-Ig in preventing dnDSA development and ABMR post-HLAi transplant. We will also assess graft function routinely and determine if Treg cells are generated with this protocol. DSAs are checked Q3M and protocol biopsy performed at 12M. The protocol is outlined in FIG. 1.

Safety determinations will be aimed at assessments of any side effects associated with clazakizumab+CTLA4-Ig administration and risk for infectious complications associated with clazakizumab therapy for desensitization of HS patients awaiting renal HLAi transplantation. Limited efficacy determinations will include assessment of the reduction of pre-existing DSA levels and inhibition of dnDSAs. This will allow prevention of ABMR (eGFR, SCr) after clazakizumab treatment. Patients will be followed and assessed for safety after treatment as well.

### Secondary Objectives

Secondary objectives will be to determine if clazakizumab+CTLA4-Ig treatment can significantly reduce or eliminate ABMR episodes and C4d deposition in incompatible allografts transplanted to highly-HLA sensitized patients. We will assess allograft function up to 12 M post-transplant, and determine renal function using SCr, MDRD GFR calculations (Schwartz equation will be used to estimate CrCI for patients under 18 years of age) as well as DSA levels. A protocol biopsy will be performed at 12M post-Clazakizumab+CTLA4-Ig therapy. We will also record any late ABMR episodes. In addition, several immunologic determinations of blood samples will be assessed at time points before and after initiation of clazakizumab therapy. These assessments will include one or more of the following: assessment of Treg cells (CD4⁺, CD25⁺, FoxP3⁺CD127^{dim}), assessment of CRP and IL-6 levels, assessment of DSAs, and the assessment of dd-cfDNA (Allosure).

An exemplary study protocol is shown in FIG. 1. These secondary end points will help us understand the biology of alloimmune responses to allografts and thereby determine the ability and mechanisms of clazakizumab's+CTLA4-Ig beneficial and potentially synergistic effects. We will also monitor viral PCRs and quantitative IgG levels as per standard-of-care.

### Inclusion Criteria

Treated patients will be 15-75 years of age at the time of screening and will include HS patients (cPRA≥50%) awaiting DD or LD kidney transplant on the UNOS list. These subjects may have a previous history of pregnancies, blood transfusion and/or renal transplant. In all instances the Subject/Parent/Guardian must be willing to participate fully with study requirements and must be able to understand and provide informed consent. These subjects should be Pneumococcal vaccinated; possess a Negative Tuberculin (ppd) placement result or negative Quantiferon TB gold results; and be EBV Igg seropositive.

### Exclusion Criteria

Treated patients will not include multi-organ transplant recipients (e.g. kidney and pancreas). Treated patients will further not include those having an Intolerability to clazakizumab or other IL-6 inhibitor therapies. Treated patients will not include lactating or pregnant females. Treated patients will not include women of child-bearing age and male partners of women of child-bearing age who are not willing or able to practice FDA-approved forms of contraception during study and for 5 months after last dose. Treated patients will not include HIV-positive subjects. Treated patients will not include subjects who test positive for HBV by HBVeAg/DNA or HCV infection [positive Anti-HCV (EIA) and confirmatory HCV RIBA]. Treated patients will not include subjects with latent or active TB. Subjects must have negative Quantiferon TB gold test result. Treated patients will not include recent recipients of any licensed or investigational live attenuated vaccine(s) within two months of the screening visit (including but not limited to any of the following: Adenovirus [Adenovirus vaccine live oral type 7], Varicella [Varivax], Hepatitis A [VAQTA], Rotavirus [Rotashield], Yellow fever [Y-F-Vax], Measles and mumps [Measles and mumps virus vaccine live], Measles, mumps, and rubella vaccine [M-M-R-II], Sabin oral polio vaccine, Rabies vaccines [IMOVAX Rabies I.D., RabAvert]). Treated patients will not include those possessing a significantly abnormal general serum screening lab result defined as an ANC<2000, platelet count<100 X 10³/ml, an SGOT or SGPT>1.5X upper limit normal. Treated patients will not include individuals deemed unable to comply with the protocol. Treated patients will not include subjects with active CMV or EBV infection as defined by CMV-specific serology (IgG or IgM) and confirmed by quantitative PCR with or without a compatible illness (Quantitative PCR cut off defined as having>50 copies of CMV or EBV DNA/PCR). Treated patients will not include those using investigational agents within 4 weeks of participation. Treated patients will not include those with a history or active Inflammatory Bowel Disease or Diverticular Disease or gastrointestinal perforation. Treated patients will not include those with recent infection (within past 6 weeks of screening) requiring any antibiotic use (oral, parenteral or topical). Treated patients will not include present or previous (within 5 years) malignancy except for basal cell carcinoma, fully excised squamous cell carcinoma of the skin or non-recurrent (within 5 years) cervical carcinoma-in-situ.

### Study Design & Methods

The trial will primarily examine the safety and tolerability of clazakizumab+CTLA4-Ig given after consented and eligible patients are identified to achieve HLAi renal transplantation at Cedars-Sinai Medical Center. 10-20 subjects (ages 15 to 75) who are highly-HLA sensitized (HS) as determined by the cPRA 50% and who are eligible for HLAi transplantation will be evaluated. All patients will be accrued from the renal transplant program at Cedars-Sinai Medical Center. Once desensitization begins, anti-HLA antibodies will be assessed which are associated with ABMR and/or graft loss. HLA antibodies will be detected using solid phase assay systems currently utilized at the Cedars-Sinai Medical Center HLA Laboratory. These anti-HLA antibodies may result naturally or from previous pregnancy, transfusions, or prior transplants. Patients treated with clazakizumab+CTLA4-Ig after HLAi transplant will have blood samples drawn which will be assayed for HLA antibodies and other moieties as well as immunologic studies.

Patients receiving HLAi transplants will initiate therapy with tacrolimus to maintain levels (7-8 ng/ml) for the first 3M post-transplant. After induction therapy with anti-CD52, patients will receive anti-IL-6 on day #7, then monthly thereafter for duration of study. At 90 days post-HLAi transplant, patients will be started on CTLA4-Ig 5 mg/kg monthly (dosing per institution standard practice) and tapered off CNI over 3M. From day 180 to the end of the study at day 365 patients will be maintained on anti-IL-6+CTLA4-Ig given monthly.

Immune monitoring in blood samples for Tregs, dd-cfDNA as well as IL-6 and CRP monitoring will be carried out at the Cedars-Sinai Transplant Immunology Laboratory.

Patients identified as study participants will be HS and listed for HLAi renal transplantation at Cedars-Sinai Medical Center which performs ˜80 HLAi renal transplants per-year. ˜100 new HS patients are referred to Cedars-Sinai Medical Center each year as potential candidates for desensitization.

Eligible patients will receive clazakizumab 25 mg 7 days post-transplant. If no safety/tolerability/efficacy issues are observed after the initial dose, patients will receive 11 additional injections Q4W. A protocol biopsy will be performed at 12M post-transplant to assess the allograft for any evidence of ABMR, including C4d staining and TG using Banff 2015 criteria. Biopsies will also be studied for molecular markers characteristic of ABMR.

Patients who develop evidence of persistent allograft dysfunction may have non-protocol biopsies for cause. Patients who receive 12 doses of clazakizumab post-transplant will receive a 12M protocol biopsy. All subjects will be evaluated on an intent-to-treat basis. Repeat laboratories will be performed at the completion of clazakizumab+CTLA4-Ig therapy to determine effect on levels and correlation with any potential events. A detailed analysis of the study is discussed below.

### Study Analysis

This single-center, Phase I/II, trial is designed to examine the safety, tolerability and limited efficacy of human clazakizumab (25 mg, subcutaneous)+CTLA4-Ig (5 mg/kg given iv) added after tacrolimus taper as per protocol (Appendix A) in 20 subjects ages 15 to 75 who are highly-HLA sensitized and are candidates for HLAi transplants at Cedars-Sinai Medical Center. Patients considered for this study are likely to have received treated with high-dose IVIG+rituximab and/or plasmapheresis prior to HLAi transplantation.

### Defining the Sensitized Patient & Criteria for HLAi Transplantation

For the purposes of this study, we define HLA sensitization as a patient awaiting kidney transplantation on the UNOS waitlist who has a cPRA of ≥50% using luminex bead technology and a history of sensitizing events (previous transplants, blood transfusions and/or pregnancies). These individuals must also have sufficient wait time on the UNOS list to allow for frequent offers and a history of positive crossmatches (DD) or an incompatible (LD) with a positive flow cytometry (FCMX) and negative complement-dependent cytotoxicity (CDC+) crossmatch. Patients proceeding to HLAi transplant will have a CDC CMX negative at 1:2 dilution, FCMX<225 channel shifts and DSAs that are at an acceptable MFI as was previously defined.

### Defining ABMR

For this study, antibody-mediated rejection (ABMR) is defined as follows: Deterioration of allograft function in a high-risk transplant recipient (i.e., sensitized patient with history of DSAs) measured by serum creatine (Cr) and estimated glomerular filtration rate (eGFR) (defined as a decline >30% from baseline); association with the presence of DSA (usually increasing in strength) measured by luminex techniques; and biopsy evidence based on BANFF 2015 grading which includes: capillaritis, inflammation and C4d deposition.

### Treatment of Allograft Rejection Episodes

Biopsy-proven rejection episodes that occur during the study are treated with “pulse” methylprednisolone (10 mg/kg/day, max 1000 mg for >100 kg for 3 days) and anti-thymocyte globulin (1.5 mg/kg daily×4) for cell-mediated rejection episodes that are unresponsive to pulse steroids. Patients experiencing recurrent ABMR episodes after study drug treatment will initially receive pulse methylprednisolone (10 mg/kg/day, max 1000 mg for >100 kg) IV daily x 3 doses then, depending on severity, IVIG 10% solution 2 gm/kg (max 140 g for >70 kg) IV X1 dose followed by rituximab (375 mg/m²) IV X1 dose. In cases where rapid deterioration of allograft function is seen and/or thrombotic microangiopathy diagnosed, the patient will receive plasma exchange X3-5 sessions followed by anti-05 (Eculizumab®) IV weekly X4 weeks (1200 mg week #1 followed by 900 mg/weekly for 3 additional weeks). Efficacy of the therapeutic regimen will be assessed by determining renal functional improvement, monitoring DSA responses and repeat allograft biopsies, if needed.

### Monitoring for AE/SAEs Post-Transplant in HS Patients

Adverse events (AEs) and serious adverse events will be monitored post-treatment with clazakizumab+CTLA4-Ig. In particular careful attention will be paid to infectious complications potentially associated with clazakizumab and CTLA4-Ig therapy.

Infectious complications associated with IVIG+rituximab desensitization and alemtuzumab induction therapy followed by maintenance therapy with tacrolimus, MMF and prednisone have been assessed by our group. Briefly, we evaluated 170 patients who were desensitized with IVIG+rituximab followed by alemtuzumab induction and maintenance therapy with tacrolimus, MMF and steroids. This was compared to a concomitant group of non-sensitized, low-risk transplants (N=191) who did not receive IVIG, rituximab or alemtuzumab (induction with IL-2R blockers).

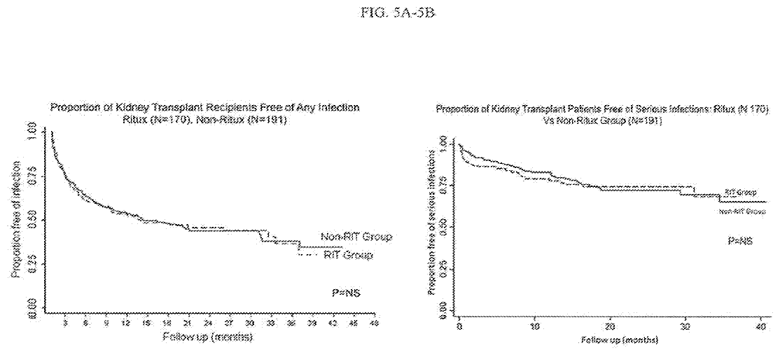

A careful analysis of all infections and serious infections that occurred over the next 4 years was compiled and is shown in FIG. 5A and 5B. Briefly, these data show that the use of this desensitization protocol followed with alemtuzumab induction does not increase the risk for common or serious infections post-transplant compared to a low risk group of patients. Serious infections were defined as any viral infection and fungal or bacterial infections requiring i.v. antibiotics or hospitalizations¹⁵. Thus risk for infections in the study group (clazakizumab) after ABMR treatment will likely be similar and comparable to non-sensitized patients. All patients entered into this study are required to be vaccinated for Streptococcus pneumoniae.

### Safety Assessments

Safety assessments will consist of monitoring and reporting all adverse events (AEs) and serious adverse events (SAEs), all events of death, and any study specific issue of concern.

### Adverse Events

An AE is any unfavorable and unintended sign, symptom, or disease temporally associated with the use of an investigational medicinal product (IMP) or other protocol-imposed intervention, regardless of attribution.

This includes the following:
AEs not previously observed in the subject that emerge during the protocol-specified AE reporting period, including signs or symptoms associated with clazakizumab infusion that were not present prior to the AE reporting period.
Complications that occur as a result of protocol-mandated interventions (e.g., renal protocol biopsy).
If applicable, AEs that occur prior to assignment of study treatment associated with medication washout, no treatment run-in, or other protocol-mandated intervention.
Preexisting medical conditions (other than the condition being studied) judged by the investigator to have worsened in severity or frequency or changed in character during the protocol-specified AE reporting period.

Serious Adverse Events—An AE should be classified as an SAE if the following criteria are met:
It results in death (i.e., the AE actually causes or leads to death).
It is life threatening (i.e., the AE, in the view of the investigator, places the subject at immediate risk of death. It does not include an AE that, had it occurred in a more severe form, might have caused death.).
It requires or prolongs inpatient hospitalization.
It results in persistent or significant disability/incapacity (i.e., the AE results in substantial disruption of the subject's ability to conduct normal life functions).
It results in a congenital anomaly/birth defect in a neonate/infant born to a mother exposed to the IMP.
It is considered a significant medical event by the investigator based on medical judgment (e.g., may jeopardize the subject or may require medical/surgical intervention to prevent one of the outcomes listed above).

### Methods and Timing for Assessing and Recording Safety Variables

The investigator is responsible for ensuring that all AEs and SAEs that are observed or reported during the study, are collected and reported.

Adverse Event Reporting Period—The study period during which all AEs and SAEs must be reported begins after informed consent is obtained and initiation of study treatment and ends 150 days after the last dose of study drug.

Reporting of Serious Adverse Events Associated with Clazakizumab® All serious adverse events (SAEs) for which there is a reasonable possibility the experience may have been caused by clazakizumab® (this applies to both expected and unexpected events) should be recorded on a MedWatch 3500A Form and MedWatch 3500A Reporting Guidelines: In addition to completing appropriate patient demographic and suspect medication information, the report should include the following information within the Event Description of the MedWatch 3500A form:
Treatment regimen (dosing frequency, combination therapy)
Protocol description (and number, if assigned)
Description of event, severity, treatment, and outcome if known
Supportive laboratory results and diagnostics
Investigator's assessment of the relationship of the adverse event to each investigational product and suspect medication

Follow-up information: Additional information may be added to a previously submitted report by any of the following methods:
Adding to the original MedWatch 3500A report and submitting it as follow-up
Adding supplemental summary information and submitting it as follow-up with the original MedWatch 3500A form
Summarizing new information and faxing it with a cover letter including subject identifiers (i.e. D.O.B. initial, subject number), protocol description and number, if assigned, suspect drug, brief adverse event description, and notation that additional or follow-up information is being submitted (The patient identifiers are important so that the new information is added to the correct initial report)

### Study Drug Relationship:

The investigator will determine which events are associated with the use of the study drugs. The causality assessment is the determination of whether there exists a reasonable possibility that the Study treatment caused or contributed to an adverse event:
Not related: Temporal relationship to Study treatment administration is missing or implausible, or there is evidence of another cause.
Possibly related: Reasonable time sequence to administration of Study treatment, but the event could also be explained by concurrent disease of other drugs or chemicals. Information on drug withdrawal may be lacking or unclear.
Definitely related: Plausible time relationship to Study treatment administration; event cannot be explained by concurrent disease or other drugs or chemicals. The response to withdrawal of the drug (dechallenge) should be clinically plausible. The event must be definitive pharmacologically or phenomenologically, using a satisfactory re-challenge procedure if necessary.

### Pregnancy Reporting

Any pregnancy occurring in a female subject or female partner of a male subject during the study or for 5 months after the last dose of study treatment should be recorded/reported on a special pregnancy form. All pregnancies will be reported within 24 hours of the Investigator being notified. In the event of a pregnancy, study treatment will be stopped. Monitoring of the pregnancy in a female subject should continue until conclusion of the pregnancy. In case of a pregnancy in the female partner of a male subject, the Investigator should obtain informed consent of the pregnant partner prior to monitoring of the pregnancy.

The outcome of all such pregnancies (including normal births) should be followed up and documented. Every effort should be made to gather information regarding the pregnancy outcome until 90 days (or otherwise as appropriate) post-partum. It is the responsibility of the Investigator, together with the appropriate support from Vitaeris, to obtain this information. Complications of pregnancy such as abortion (spontaneous or induced), premature birth, or congenital abnormality are considered SAEs and will be reported.

### Infection Prophylaxis Protocols and Viral Monitoring Post-Transplant

All study patients, regardless of their cytomegalovirus (CMV) status, will receive IV ganciclovir while inpatients and valganciclovir as outpatients for 6 months post kidney transplant, with dose adjustments for renal function. Fungal prophylaxis was accomplished with fluconazole 100 mg daily for 1 month post-transplant. Pneumocystis jirovecii pneumonia and bacterial prophylaxis is accomplished with trimethoprim 80 mg and sulfamethoxazole 400 mg daily for 6-12 months post-transplant.

Viral polymerase chain reaction assays for CMV, Epstein Barr virus, Parvovirus B-19, Polyoma virus BK and JC will be performed on study patients monthly for 6 months post-transplantation. Methodologies used for monitoring viral replication have been described previously.

### Clazakizumab

Dosing Clazakizumab will be administered at a dose of 25 mg SC Q4W starting at Day 7 post initial desensitization with PLEX+IVIG, and repeated at Q4W intervals for a maximum of 6 doses. If patient get transplanted they will receive clazakizumab monthly starting post-transplant day 5 (after IVIG doses).

### Product Description, Storage and Administration Instructions

Clazakizumab will be provided by Vitaeris Inc.; Active ingredient: Genetically engineered humanized anti-IL-6 mAb; Strength: 25 mg/mL; Excipients: L-histidine, L-histidine monohydrochloride, sorbitol, polysorbate-80, and water for injection; Appearance:

Clear to slightly opaque, colorless to dark yellow-colored solution; Dosage form: Single-dose vials (25 mg/mL) for injection.; Manufacturer: Ajinomoto Althea, San Diego Calif.; Clazakizumab vials should be stored at ≤−20° C. (−4° F.) with protection from light.

The drug product will be administered undiluted at a concentration of 25 mg/mL. Prepared syringes may be stored for up to 24 hours in a refrigerator, 2°-8° C. (36°-46° F.) ≤−20° C. (−4° F.), and up to 4 hours of the 24 hours may be at room temperature, 15°-25° C. (59°-77° F.). The prepared syringes should be protected from light.

Prior to administration, clazakizumab should reach room temperature by storing unrefrigerated for 30 to 60 minutes before use.

Clazakizumab will not be used after the expiry date (EXP) shown on the kit or vial.

There are no specific antidotes or measures to take in the event of an overdose of clazakizumab injection. Subjects should be treated with the appropriate supportive care.

A number of measures will be taken to ensure the safety of patients participating in this study. These measures will be addressed through exclusion criteria and routine monitoring as follows: Patients enrolled in this study will be evaluated clinically and with standard laboratory tests before and during their participation in this study. Safety evaluations will consist of medical interviews, recording of adverse events, physical examinations, blood pressure, and laboratory measurements. Subjects will be evaluated for adverse events (all grades), serious adverse events, and adverse events requiring study drug interruption or discontinuation at each study visit for the duration of their participation in the study.

### Opportunistic Infections and Serious Infections

Physicians should exercise caution when considering the use of clazakizumab in patients with a history of recurring infection or with underlying conditions (e.g., diabetes) which may predispose patients to infections. Clazakizumab should not be administered in patients with active infection. The effects of clazakizumab on CRP, neutrophils, and the signs and symptoms of infection should be considered when evaluating a patient for a potential infection.

Vigilance for timely detection of serious infection is recommended for patients receiving biologic agents for treatment of moderate to severe RA as signs and symptoms of acute inflammation may be lessened due to suppression of the acute phase reaction. Patients must be instructed to contact their physician immediately when any symptoms suggesting infection appear, in order to assure rapid evaluation and appropriate treatment.

If a patient develops a serious infection, administration of clazakizumab is to be interrupted until the infection is controlled. The clinician should consider the benefit-risk before resuming treatment with clazakizumab.

### Gastrointestinal Perforations

Timely diagnosis and appropriate treatment may reduce the potential for complications of diverticulitis and thus reduce the risk of GI perforations. Therefore, patients should be made aware of the symptomatology potentially indicative of diverticular disease, and they should be instructed to alert their healthcare provider as soon as possible if these symptoms arise. In patients with a history of symptomatic diverticulosis, diverticulitis or chronic ulcerative lower GI disease such as Crohn's disease, ulcerative colitis or other chronic lower GI conditions that might predispose to perforations, the clinician should consider the benefit-risk before using clazakizumab. Discontinuation of clazakizumab is recommended for patients who develop GI perforations.

### Demyelinating Disorders

The impact of treatment with clazakizumab on demyelinating disorders is not known; events were rarely reported. Patients should be closely monitored for signs and symptoms potentially indicative of central demyelinating disorders. Physicians should exercise caution in considering the use of clazakizumab in patients with pre-existing or recent onset demyelinating disorders. Treatment with clazakizumab should be interrupted during assessment of a potential demyelination event and only resumed if the benefit of continuing study drug is favorable.

### Hematologic Abnormalities and Bleeding Events

Decreases in neutrophil and platelet counts have been observed following treatment with clazakizumab in combination with MTX (methotrexate). In addition, there may be an increased risk of neutropenia in patients who have previously been treated with a TNF antagonist.

For patients with concomitant medications associated with hematologic toxicity, the reduction or interruption of the suspected medication is recommended prior to modifying clazakizumab.

TABLE 1
Neutropenia Risk Mitigation

| ANC (cells/mm³) | Action |
|---|---|
| >1000 | Maintain dose. |
| 500-1000 | Interrupt clazakizumab dosing. |
| | When ANC increases to >1000, resume |
| | clazakizumab 25 mg SQ monthly |
| <500 | Discontinue clazakizumab. |

ANC = absolute neutrophil count

TABLE 2
Thrombocytopenia Risk Mitigation

| Platelet count | |
|---|---|
| (cells/mm³) | Action |
| >100,000 | Maintain dose. |
| 50,000-100,000 | Interrupt clazakizumab dosing. |
| | When platelet count increases |
| | to >100,000, resume |
| | Clazakizumab at 25 mg SQ monthly |
| <50,000 | Discontinue clazakizumab. |

### Elevated Liver Enzymes and Hepatic Events

Elevations in ALT and AST have been observed during treatment with the study medications

TABLE 3

| Lab Value | Action |
|---|---|
| >1 to 3x ULN | Dose modify concomitant transplant |
| | immunosuppressive drugs if appropriate |
| | For persistent increases in this range, |
| | interrupt clazakizumab until ALT/AST |
| | have normalized |
| | Restart with 25 mg SQ monthly as |
| | clinically appropriate |
| >3 to 5x ULN | Interrupt clazakizumab dosing |
| (confirmed by | until <3x ULN and follow |
| repeat testing) | recommendations above for >1 to 3x ULN |
| | For persistent increases >3x ULN, |
| | discontinue clazakizumab |
| | Discontinue clazakizumab if total |
| | bilirubin >2x ULN |
| >5x ULN | Discontinue clazakizumab |

### Cardiovascular Events and Elevated Lipids

Patients with RA have an increased risk for cardiovascular disorders, therefore, risk factors for cardiovascular disease (e.g., hypertension, hyperlipidemia) should be managed as part of their standard of care. See section on Drug Interactions.

For patients with LDL cholesterol 160 mg/dL, it is strongly recommended that investigators advise therapeutic lifestyle changes that may include initiation lipid-lowering agents. Lipid-lowering agents should also be considered for patients with lower LDL cholesterol levels as part of their therapeutic lifestyle changes depending on their overall risk as defined in NCEP ATP III or other national guidelines.

### Malignancies

The impact of immunosuppression on the development of malignancies is not known, however an increased rate of some malignancies, notably lymphoma, has been observed in RA patients. Although no imbalance of malignancies was observed in clinical trials of Clazakizumab, malignancies have been identified as a concern for other biologics. It is recognized that identification of such events in clazakizumab-treated patients may require a longer period of surveillance. Clazakizumab should be discontinued in patients with malignancies (with the exception of local basal or squamous cell carcinoma of the skin that is completely excised with free margins).

### Hypersensitivity or Anaphylaxis

An infusion/dose reaction is defined as an adverse event occurring during and within 24 hours after the infusion or subcutaneous injection of clazakizumab. This may include hypersensitivity reactions or anaphylactic reactions. Signs of a possible hypersensitivity reaction include but are not limited to:

fever, chills, pruritus, urticaria, angioedema, and skin rash.

cardiopulmonary reactions, including chest pain, dyspnea, hypotension or hypertension.

Healthcare professionals administering clazakizumab should be trained in the appropriate administrative procedures, be able to recognize the symptoms associated with potential anaphylactic or hypersensitivity reactions, and have the appropriate medication available for immediate use in case of anaphylaxis or hypersensitivity reaction during or after administration of clazakizumab. Healthcare professionals should also instruct patients to seek medical attention if they experience symptoms of a hypersensitivity reaction outside of the clinic.

If a patient has symptoms of anaphylaxis or serious hypersensitivity, or requires an interruption of the study drug because of symptoms of anaphylaxis or hypersensitivity, administration of clazakizumab must be discontinued permanently. The patient should be treated according to the standard of care for management of the hypersensitivity reaction. A blood sample for the presence of anti-clazakizumab antibodies should be obtained.

Clazakizumab should not be administered to subjects who have had any previous allergic reactions to monoclonal antibodies. To date, no infusion reactions have been associated with clazakizumab administered by IV infusion. Injection site reactions have been reported with SC administration. Reactions have been mild or moderate and have resolved without treatment. Both allergic reactions and injection site reactions should be treated with standard of care. Subjects who have developed significant allergic reaction to study drugs should not be re-challenged.

### Viral Reactivation

Though rarely reported within the clazakizumab program due to exclusion criteria at study entry, reactivation of viral and other serious infections (e.g. EBV or TB) has been observed with biologic therapies.

### Drug Interaction

The formation of CYP450 enzymes may be suppressed by increased levels of cytokines (e.g., IL-6) during chronic inflammation. Therefore, it is expected that for molecules that antagonize cytokine activity, such as clazakizumab, the formation of CYP450 enzymes could be normalized. When starting or stopping therapy with clazakizumab, patients taking medications which are individually dose-adjusted and metabolized via CYP450, 3A4, 1A2, or 2C9 (e.g., atorvastatin, calcium channel blockers, theophylline, warfarin, phenytoin, cyclosporine, or benzodiazepines) should be monitored as doses may need to be adjusted to maintain their therapeutic effect. Given its long elimination half-life (t1/2) of about 30 days the effect of clazakizumab on CYP450 enzyme activity may persist for several weeks after stopping therapy.

### Pregnancies and Women of Child Bearing Potential

There are no adequate well-controlled studies in pregnant or lactating women. In nonclinical studies, an increase in the number of monkeys with retention of the placenta at parturition was observed at clazakizumab doses corresponding to 11 and 110 times the planned human dose of 50 mg. In 3 of the 5 monkeys with retained placentas, the resulting excessive uterine hemorrhage led to moribund status in the mothers.

Three pregnancies have been reported to date in subjects taking clazakizumab. The outcomes included one spontaneous abortion (outcome unknown for the other 2 pregnancies).

All subjects of child bearing potential being treated with clazakizumab (and their partners) must be informed of this risk, and use highly effective birth control. Administration of clazakizumab may decrease the efficacy of hormonal oral contraceptives. For this study, under no circumstances shall clazakizumab injection be administered to women known to be pregnant or lactating. All pregnancies must be reported to Vitaeris within 24 hours and in accordance with SAE reporting procedures.

### Therapy Stopping Points

As indicated previously, the study will be halted and re-evaluated by the Data and Safety Monitoring Board (DSMB) if any patient in the study group develops SAEs or evidence of severe infusion related or infectious complications. In addition, we will note if patients develop severe ABMR after transplantation with clazakizumab+CTLA4-Ig and fail to respond to this treatment and require reinstitution of standard of care treatments (IVIG+rituximab +/− PLEX). If more than 2 patients fail to show improvements in ABMR, the study will be halted and reassessment of the study goals and complications will be done and discussed with Vitaeris (collaborator), the DSMB and FDA prior to reinitiating the study.

### Example 3

### Effect of Clazakizumab Administration on HLA antibodies in Transplant Recipients

Patients entered into the afore-described clazakizumab desensitization trial (NCT03380962) where 75% were in the 99-100% Panel Reactive Antibody (PRA) range received clazakizumab 25 mg subcutaneously monthly for up to 6 months. If transplanted, they received continued clazakizumab for up to 6 M post-transplant. This group of patients are exceedingly difficult to transplant due to high likelihood of severe antibody mediated rejection and graft loss post-transplant. Antibody levels, pre-post-transplant and Treg cells were monitored at concomitant time points.

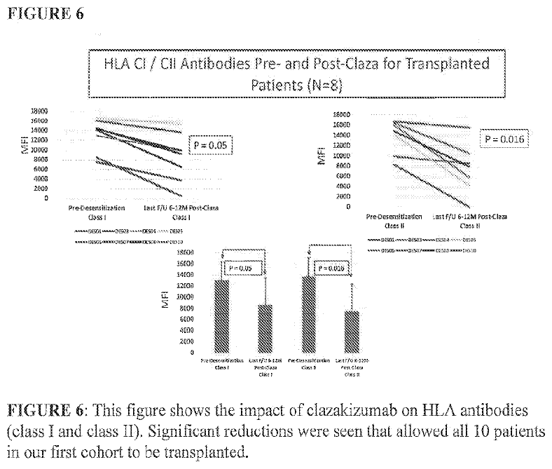

FIG. 6 shows the impact of clazakizumab on HLA antibodies (class I and class II) pre- and post-clazakizumab administration in these transplant patients. As can be seen from the results therein there were significant reductions in both class I and class II HLA antibodies in all patients who were treated with clazakizumab. These reductions were deemed sufficient to permit all of these patients to be transplanted.

### Example 4

### Effect of Clazakizumab Administration on Donor Specific Antibodies in Transplant Recipients

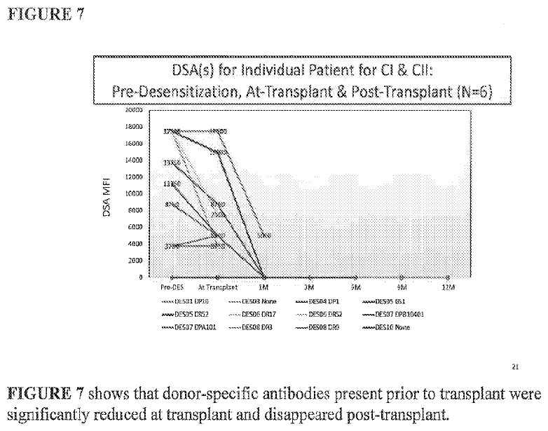

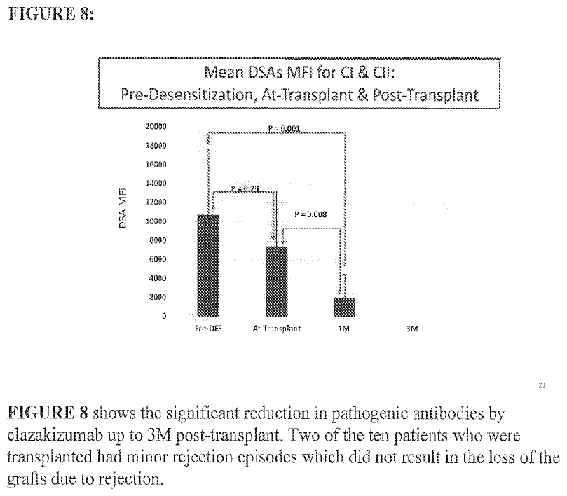

Transplant patients who were treated as described in the previous example were also monitored to assess the effect of clazakizumab on donor specific antibodies. Particularly, levels of donor specific antibodies were monitored in transplant patient samples pre- and post-transplant. These results are shown in FIG. 7 and FIG. 8.

FIG. 7 shows that donor-specific antibodies which were present prior to transplant were significantly reduced at transplant and disappeared post-transplant in all transplant patients who received clazakizumab.

FIG. 8 shows that a significant reduction in pathogenic antibodies was observed in these same transplant patients who were treated with clazakizumab at least up to 3 months post-transplant. Also, while 2 of 10 transplant patients had rejection episodes, importantly no grafts were lost due to rejection.

### Example 5

### Clazakizumab Induces FoxP3⁺ Tregs in Highly HLA Sensitized Patients Desensitized for HLAi Transplantation 180 Days post-Transplant

As part of a phase I/II trial of clazakizumab for desensitization, HLA sensitized patients received anti-IL-6, 25 mg SC monthly×6 doses with monitoring of HLA antibody levels and Tregs. Patients were treated pre- and post-transplant with clazakizumab. Transplanted patients received monthly clazakizumab 25 mg subcutaneously starting 5-7 days post-transplant for 12 months. Tregs were determined by flow cytometry as CD4⁺, CD25⁺, CD127^{dim}, FoxP3⁺ cell populations in CD4⁺ cells. Determinations were made at baseline, at transplantation and day 180 post-transplant.

Nine patients were transplanted. All patients had previous transplants; 78% had cPRA 99-100%, 67% were B-cell FCMX⁺ and class II DSA⁺ at the time of transplant. Mean MFI for HLA cl and cll were: pre-desensitization vs. post claza: cl 13062±3123 vs. 8585±4597 (p=0.05) and cll 13519±2966 vs. 8344±4836 (p=0.03).

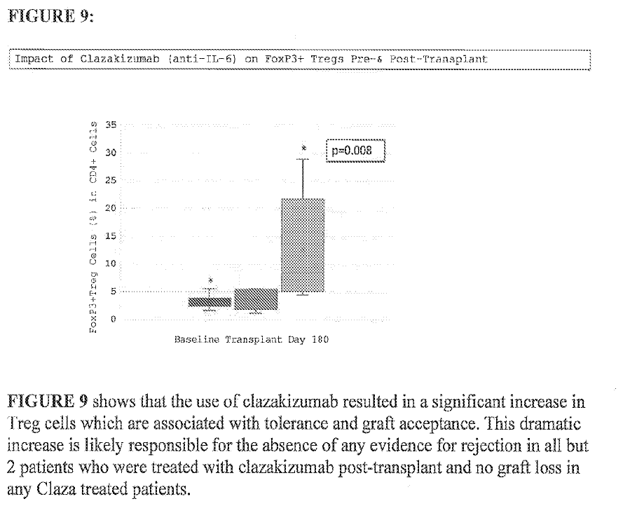

All DSA+ patients were negative by day 180 post-transplant. Mean Treg values at baseline v. at transplant were not statistically different (3.2+1.09% v.3.5+1.75%, p=NS). By contrast Treg values were significantly different at day 180 post-transplant (3.2+1.09% v.3.5+1.75% v. 12.6+9.3%, p=0.008) These results are contained in FIG. 9.

These results show that clazakizumab desensitization reduced HLA cl/cll antibodies and allowed 9/10 highly sensitized patients to receive transplants. In addition, a dramatic increase in Treg cells at day 180 post-transplant was seen while patients were still on anti-IL-6 therapy suggesting that anti-IL-6 may shift CD4+T-cell responses to a Treg profile. This may have therapeutic implications for modifying baseline immunosuppression post-transplant.

The results in the previous examples are compelling and substantiate that the administration of clazakizumab to transplant recipients may be used to effectively prevent immune activation events and rejection for a prolonged duration after transplant, potentially indefinitely. These results are further compelling because the patient population who was treated with clazakizumab comprise are part of a population group well known to be extremely prone to transplant rejection and graft loss.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of immunosuppression or immunomodulation in a solid organ transplant recipient, comprising:
administering an IL-6 inhibitor or IL-6R inhibitor, or both to the recipient; and
administering CTLA-4 or a CTLA4 fusion protein, to the recipient.

2. The method of claim 1, wherein the CTLA-4 or CTLA-4 fusion protein, is CTLA 4 Ig.

3. The method of claim 1, wherein the CTLA-4 or a CTLA4 fusion protein comprises:
(i) belatacept;
(ii) belatacept biosimilar;
(iii) abatacept; or
(iv) any combination of the foregoing.

4. The method of claim 1, wherein the IL-6 inhibitor comprises:
(i) Siltuximab;
(ii) Clazakizumab;
(iii) Olokizumab;
(iv) sirukumab;
(v) FB-704A;
(vi) ARGX-109;
(vii) EBI-031;
(viii) AH-65;
(ix) SL-1026;
(x) ES-306;
(xi) AM-201;
(xii) lsilimomab;
(xiii) MAb 1339;
(xiv) a salt of any of the foregoing; or
(xv) any combination of the foregoing.

5. The method of claim 1, wherein the IL-6-R inhibitor comprises:
(i) Tocilizumab;
(ii) Sarilumab;
(iii) BAT-1806;
(iv) Satralizumab;
(v) Vobarilizumab;
(vi) Olamkicept;
(vii) BCD-089;
(viii) CMAB-806;
(ix) QX-003S;
(x) HS-628;
(xi) LusiNEX;
(xii) MT-6194;
(xiii) TZLS-501;
(xiv) a salt of any of the foregoing; or
(xv) any combination of the foregoing.

6. The method of claim 1, wherein:
(i) a first dose of the IL-6 inhibitor and/or the IL-6R inhibitor is administered 5-10 days after transplantation; or
(ii) the IL-6 inhibitor and/or the IL-6R inhibitor is administered every 20-40 days after transplantation.

7. (canceled)

8. The method of claim 1, wherein:
(i) the IL-6 inhibitor and/or the IL-6R inhibitor is administered for at least one year;
(ii) a first dose of the CTLA-4 or CTLA-4 fusion protein is administered 75-105 days after transplantation;
(iii) the CTLA-4 or CTLA-4 fusion protein is administered every 20-40 days after the first dose of the CTLA-4 or CTLA-4 fusion protein; and/or
(iv) the CTLA-4 or CTLA-4 fusion protein is administered for at least one year.

9. (canceled)

10. (canceled)

11. (canceled)

12. The method of claim 1, further comprising administering a calcineurin inhibitor.

13. The method of claim 12, wherein the calcineurin inhibitor is selected from
(i) cyclosporine;
(ii) modified cyclosporine (modified);
(iii) Voclosporin;
(iv) Pimecrolimus;
(v) tacrolimus;
(vi) a salt of any of the foregoing;
(vii) any combination of the foregoing.

14. The method of claim 12, wherein the calcineurin inhibitor is administered 0-3 days after transplantation and for 75-105 days after transplantation.

15. (canceled)

16. The method of claim 1, wherein the method does not comprise administering a calcineurin inhibitor.

17. The method of claim 12, wherein:
(i) the IL-6 inhibitor is clazakizumab; the CTLA4-Ig is belatacept, belatacept biosimilar, or abatacept; and the calcineurin inhibitor administered, is selected from cyclosporine, cyclosporine (modified), voclosporin, pimecrolimus, tacrolimus, a salt of any of the foregoing, or any combination of the foregoing calcineurin inhibitors;
(ii) the IL-6 inhibitor is Siltuximab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(iii) the IL-6 inhibitor is olokizumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(iv) the IL-6 inhibitor is sirukumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(v) the IL-6 inhibitor is FB-704A; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(vi) the IL-6 inhibitor is ARGX-109; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(vii) the IL-6 inhibitor is EBI-031; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(viii) the IL-6 inhibitor is AH-65; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(ix) the IL-6 inhibitor is ES-306; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(x) the IL-6 inhibitor is AM-201; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(xi) the IL-6 inhibitor is lsilimomab (also known as “B-E8”); the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(xii) the IL-6 inhibitor is MAb 1339 (a high affinity variant of Elsilimomab); the CTLA4-Ig is belatacept, belatacept biosimilar, or abatacept; and the calcineurin inhibitor is selected from the group consisting of cyclosporine, cyclosporine (modified), voclosporin, pimecrolimus, tacrolimus, a salt of any of the foregoing, or any combination of the foregoing calcineurin inhibitors;
(xiii) the IL-6 inhibitor is a salt of any of the foregoing; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors; or
(xiv) the IL-6 inhibitor is any combination of the foregoing IL-6 inhibitors; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors.

18. The method of claim 12, wherein:
(i) the IL-6-R inhibitor is Tocilizumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(ii) the IL-6-R inhibitor is Sarilumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(iii) the IL-6-R inhibitor is tocilizumab biosimilar (BAT-1806); the CTLA4-Ig is belatacept, belatacept biosimilar, or abatacept; and the calcineurin inhibitor is selected from cyclosporine, cyclosporine, cyclosporine (modified), voclosporin, pimecrolimus, tacrolimus, a salt of any of the foregoing, or any combination of the foregoing calcineurin inhibitors;
(iv) the IL-6-R inhibitor is Satralizumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(v) the IL-6-R inhibitor is Vobarilizumab; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(vi) the IL-6-R inhibitor is Olamkicept; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(vii) the IL-6-R inhibitor is BCD-089; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(viii) the IL-6-R inhibitor is CMAB-806, the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(ix) the IL-6-R inhibitor is tocilizumab biosimilar (QX-003S); the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(x) the IL-6-R inhibitor is HS-628; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(xi) the IL-6-R inhibitor is tocilizumab biosimilar (LusiNEX); the CTLA4-Ig is belatacept, belatacept biosimilar, or abatacept; and the calcineurin inhibitor is selected from the cyclosporine, cyclosporine (modified), voclosporin, pimecrolimus, tacrolimus, a salt of any of the foregoing, or any combination of the foregoing calcineurin inhibitors;
(xii) the IL-6-R inhibitor is MT-6194; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors;
(xiii) the IL-6-R inhibitor is TZLS-501; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors; or
(xiv) the IL-6-R inhibitor is a salt of any of the foregoing Il-6R inhibitors; the CTLA4-Ig is belatacept, belatacept biosimilar, or abatacept; and the calcineurin inhibitor is selected from cyclosporine, cyclosporine (modified), voclosporin, pimecrolimus, tacrolimus, a salt of any of the foregoing, or any combination of the foregoing calcineurin inhibitors.

19. The method of claim 1, wherein the recipient is a highly-HLA sensitized recipient.

20. The method of claim 1, wherein the solid organ comprises a kidney, heart, lung, pancreas, liver, or a combination of any of the foregoing.

21. The method of claim 20, wherein the solid organ comprises a kidney.

22. The method of claim 19, wherein the solid organ is human leukocyte antigen (HLA) incompatible.

23. The method of claim 1, wherein ABMR, dnDSA development, and/or allosensitization is prevented or reduced.

24. (canceled)

25. (canceled)

26. (canceled)

27. A method of immunosuppression or immunomodulation in a solid organ transplant recipient, comprising:
administering a first dose of clazakizumab 5-10 days after transplantation to the recipient;
administering a subsequent dose of clazakizumab every 20-40 days to the recipient;
administering a first dose of belatacept 75-105 days after transplantation to the recipient; and
administering a subsequent dose of belatacept every 20-40 days to the recipient.

28. (canceled)

29. The method of claim 277, wherein the method does not comprise administering a calcineurin inhibitor after 75-105, 75-95, 75-85, or 95-105 days after transplantation.

30. The method of claim 27, wherein ANC (absolute neutrophil count) (cells/mm³) is monitored and dosing of clazakizumab is:
(i) maintained if ANC is >1000;
(ii) interrupted if ANC ranges from 500-1000; or
(iii) discontinued if ANC is less than 500.

31. (canceled)

32. The method of claim 30, part (ii), wherein ANC (absolute neutrophil count) (cells/mm³) is monitored and dosing of clazakizumab is resumed if ANC increases to 1000 or more.

33. (canceled)

34. The method of claim 27, wherein platelet count (cells/mm³) is monitored and dosing of clazakizumab is:
(i) maintained if platelet count (cells/mm³) is >100,000;
(ii) interrupted if platelet count (cells/mm³) is within the range of 50,000-100,000; or
(iii) discontinued if platelet count (cells/mm³) is <50,000.

35. (canceled)

36. The method of claim 34, part (ii), wherein platelet count (cells/mm³) is monitored and dosing of clazakizumab is resumed if platelet count (cells/mm³) increases to >100,000.

37. (canceled)

38. The method of claim 27, wherein liver enzyme levels (ALT and AST) are monitored and:
(i) dosing of clazakizumab is maintained if elevations in liver enzymes (ALT and AST) do not increase to >3 to 5× ULN or do not persist in the range (>1 to 3× ULN);
(ii) if liver enzyme levels increase >1 to 3× ULN, the dosage of concomitant transplant immunosuppressive drugs the subject is receiving is modified (increased) and/or at least one other concomitant transplant immunosuppressive drug is administered; or
(iii) if elevations in liver enzymes (ALT and AST) increase in the recipient to within the range of >3 to 5× ULN, dosing of clazakizumab in the recipient is interrupted until liver enzymes are <3× ULN.

39. (canceled)

40. The method of claim 38, part (ii), wherein if said elevations in liver enzymes (ALT and AST) persist in the range (>1 to 3× ULN) then dosing of clazakizumab is interrupted until ALT/AST levels have normalized.

41. (canceled)

42. The method of claim 38, part (iii), wherein when elevations in liver enzymes (ALT and AST) are reduced to within the range of >1 to 3× ULN,. the dosage of concomitant transplant immunosuppressive drugs the subject is receiving is increased and/or at least one other concomitant transplant immunosuppressive drug is administered.

43. The method of claim 27, wherein liver enzyme and total bilirubin in the recipient are monitored and if the recipient experiences: (i) persistent increases in liver enzymes of >3× ULN; (ii) liver enzyme increases of >5× ULN; and/or (iii) if total bilirubin is >2 × ULN, then administration of clazakizumab is discontinued in the recipient.

44. The method of claim 27, wherein the method comprises 2 or optionally 3 distinct phases: (i) an initial phase where clazakizumab is administered, without any belatacept, (2) a second phase after the initial phase where both clazakizumab and Igbelatacept are administered, and optionally (3) a third phase after (2) where only clazakizumab or belatacept is administered.

45. The method of claim 44 wherein the method does not include administration of a calcineurin inhibitor.

46. The method of claim 44; (i) Phase 1 comprises administering a first dose of clazakizumab 5-10 days after transplantation and another dose every 20-40 days; and (ii) Phase 2 comprises administering a first dose of belatacept 75-105 days after transplantation and administering a subsequent dose of belatacept every 20 40days and further includes administration of clazakizumab every 20-40 days.

47. The method of claim 44, wherein: (i) Phase 1 comprises administering a first dose of clazakizumab 7 days after transplantation and administering a subsequent dose of clazakizumab every 30 days; and (ii) Phase 2 comprises administering a first dose of belatacept 90 days after transplantation and administering a subsequent dose of belatacept every 30 days.

48. The method of claim 44, which does not include administering a calcineurin inhibitor after 75-105 days after transplantation.

49. The method of claim 44, wherein the further comprises administration of a calcineurin inhibitor, optionally in phase (2) and/or (3).

50. The method of claim 1 wherein the anti-IL-6 and/or anti-IL-6R inhibitor comprises clazakizumab and the CTLA-4 comprises CTLA 4 Ig.

51. The method of claim 49, wherein the calcineurin inhibitor is selected from cyclosporine, cyclosporine (modified), tacrolimus, a salt of any of the foregoing, or any combination of the foregoing calcineurin inhibitors.

52. A kit for immunosuppression or immunomodulation, comprising:
an IL-6 inhibitor or IL-6R inhibitor, or both; and
a CTLA4-Ig; and
instructions for using the IL-6 inhibitor or IL-6R inhibitor, or both, and the CTLA4-Ig for immunosuppression or immunomodulation in a solid organ transplant recipient.

53. The kit of claim 52, wherein the CTLA4-Ig comprises:
(i) belatacept,
(ii) belatacept biosimilar,
(iii) abatacept, or
(iv) any combination of the foregoing.

54. The kit of claim 52, wherein the CTLA4-Ig comprises belatacept.

55. The kit of claim 52, wherein the IL-6 inhibitor comprises:
(i) Siltuximab;
(ii) Clazakizumab,
(iii) olokizumab,
(iv) sirukumab,
(v) FB-704A,
(vi) ARGX-109,
(vii) EBI-031,
(viii) AH-65,
(ix) SL-1026,
(x) ES-306,
(xi) AM-201,
(xii) lsilimomab,
(xiii) MAb 1339;
(xiv) a salt of any of the foregoing; or
(xv) any combination of the foregoing.

56. (canceled)

57. The kit of claim 52, further comprising a calcineurin inhibitor.
